(19) United States
(12) Patent Application Publication
(10) Pub. No.: US 2021/0196627 A1
(43) Pub. Date: Jul. 1, 2021

(54) SYSTEMS FOR ENTERIC DELIVERY OF THERAPEUTIC AGENTS

(71) Applicant: Lyndra, Inc., Watertown, MA (US)

(72) Inventors: Tyler GRANT, Arlington, MA (US); Rosemary KANASTY, Cambridge, MA (US); David ALTREUTER, Wayland, MA (US); Andrew BELLINGER, Wellesley, MA (US); Alisha WEIGHT, Cambridge, MA (US); Stephen ZALE, Hopkinton, MA (US); Susan LOW, Pepperell, MA (US)

(21) Appl. No.: 17/268,405

(22) PCT Filed: Aug. 13, 2019
(86) PCT No.: PCT/US2019/046369

§ 371 Date: Feb. 12, 2021

Related U.S. Application Data
(60) Provisional application No. 62/764,917, filed on Aug. 15, 2018.

(51) Int. Cl. A61K 9/00; A61K 9/48

(52) U.S. Cl. CPC A61K 9/0065; A61K 9/4808; A61K 9/4891

(57) ABSTRACT

Described herein are systems for the enteric delivery of therapeutic agents, and methods of administering a therapeutic agent to a patient by orally administering an enteric delivery system. The enteric deliver system includes one or more carrier members comprising a carrier polymer and a therapeutic agent, and the system is configurable in a compacted configuration and an expanded configuration, and is sized to maintain contact with the intestinal wall of the small intestine by applying an outwardly directed pressure to the intestinal wall and transport at least a portion of the therapeutic agent across the enteric mucosa of the small intestine.

### CROSS-REFERENCE TO RELATED APPLICATIONS

This application claims priority benefit of U.S. Provisional Patent Application No. 62/764,917 filed Aug. 15, 2018. The entire contents of that application are hereby incorporated by reference herein.

### FIELD OF THE INVENTION

The present disclosure relates to systems that are configured for sustained release and enteric delivery of therapeutic agents, such as biological macromolecules, and methods of using and making such systems.

### BACKGROUND OF THE INVENTION

Administration of many therapeutic agents, particularly biological macromolecules such as proteins or oligonucleotides, relies on subcutaneous or intravenous administration. Orally administered formulations of therapeutics are highly desirable to increase ease of administration and compliance compared to injectable administrations. However, oral administration is generally ineffective due to the acidic gastric environment and poor adsorption through the intestinal wall. Enteric formulations of certain therapeutic agents have been developed for orally administered sustained release in the small intestine, but such formulations are generally limited to hydrophobic small molecule agents.

Previous attempts to enhance enteric delivery of biomolecules have included nanoparticle delivery and the use of needles or microneedles to pierce the intestinal wall. Such methods often have inconsistent of low efficiency of drug uptake, or do not allow for sustained delivery.

### SUMMARY OF THE INVENTION

Described herein are systems for the enteric delivery of therapeutic agents. Also described herein are therapeutic dosage forms that include a capsule encapsulating the any one of the enteric delivery systems described herein. Further described are methods of administering a therapeutic agent to a patient by orally administering an enteric delivery system.

Described herein is a system for enteric delivery of a therapeutic drug, comprising: one or more carrier members comprising a carrier polymer and a therapeutic agent, the system configurable in a compacted configuration and an expanded configuration, wherein the system is configured to (1) expand from the compacted configuration to the expanded configuration within the small intestine, or (2) expand from the compacted configuration to the expanded configuration within the stomach and pass through the pylorus without substantial release of the therapeutic agent until reaching the small intestine; wherein the system is sized to maintain contact with the intestinal wall of the small intestine by applying an outwardly directed pressure to the intestinal wall and transport at least a portion of the therapeutic agent across the enteric mucosa of the small intestine; and wherein at least a portion of the system loses structural integrity after a period of time within the small intestine to release the outwardly directed pressure.

In some embodiments, the system is configured to expand from the compacted configuration to the expanded configuration within the small intestine.

In some embodiments, the system is configured to expand from the compacted configuration to the expanded configuration within the stomach and pass through the pylorus without substantial release of the therapeutic agent until reaching the small intestine.

In some embodiments, the one or more carrier members comprise a coating comprising the therapeutic agent.

In some embodiments, the coating further comprises a permeability enhancing agent.

In some embodiments, the therapeutic agent is loaded into the carrier polymer.

In some embodiments, permeability enhancing agent is loaded into the carrier polymer. In some embodiments, the permeability enhancing agent is a muco-adhesive agent or a muco-permeating agent. In some embodiments, the permeability enhancing agent is a fatty acid, a bile salt, chitosan, a thiolated polymer, or a cell penetrating peptide.

In some embodiments, the outwardly directed pressure is released after about 1 hour to about 72 hours after the system enters the small intestine. In some embodiments, release of the outwardly directed pressure allows for passage of the carrier members through the small intestine.

In some embodiments, the system is configured to transport the therapeutic agent across the enteric mucosa for about 1 hour to about 72 hours.

In some embodiments, the wherein the system is sized maintain contact with the intestinal wall of the duodenum by applying an outwardly directed pressure to the intestinal wall of the duodenum and transport at least a portion of the therapeutic agent across the enteric mucosa of the duodenum.

In some embodiments, the one or more carrier members comprise a hollow core. In some embodiments, the one or more carrier members comprise a solid core.

In some embodiments, the one or more carrier members are configured to lose structural integrity after a period of time within the small intestine to release the outwardly directed pressure.

In some embodiments, the one or more carrier members are configured to lose structural integrity through erosion, degradation, or softening of the one or more carrier members.

In some embodiments, the one or more carrier members are arranged in a ring shape.

In some embodiments, the system further includes one or more linkers that join the one or more carrier members to form the ring shape, the one or more linkers comprising a polymer configured to lose structural integrity after a period of time in the small intestine.

In some embodiments, the therapeutic drug is within a coating on or in an outer portion of the ring shape, but not on or in an inner portion of the ring shape.

In some embodiments, the system further comprises an elastomeric central member attached to a plurality of arms radiating outwardly from the central member when the system is in an extended configuration, the arms comprising one or more carrier members.

In some embodiments, the therapeutic drug of the system is preferentially disposed on or within distal ends of the arms relative to the elastomeric central member.

In some embodiments, the elastomeric central member comprises a polymer configured to lose structural integrity after a period of time in the small intestine. In some embodiments, the elastomeric central member is configured to lose structural integrity through erosion, degradation, or softening of the elastomeric central member.

In some embodiments, the elastomeric central member is joined to the arms through one or more linkers comprising a polymer configured to lose structural integrity after a period of time in the small intestine.

In some embodiments, the system loses structural integrity through erosion, degradation, or softening of the one or more linkers.

In some embodiments, the carrier members have a circular, elliptical, or teardrop cross section.

In some embodiments, the therapeutic agent is a polypeptide or a polynucleotide. In some embodiments, the therapeutic agent is a polypeptide comprising 10 or more amino acids. In some embodiments, the therapeutic agent is a polynucleotide comprising 10 or more nucleotides.

In some embodiments, the small intestine is a small intestine of a human.

In some embodiments, the system is coated with a protective coating. In some embodiments, the protective coating is an enteric coating. In some embodiments, the system is further coated with a reverse-enteric coating.

Also described herein is a therapeutic dosage form comprising a capsule encapsulating any of the above-described systems. In some embodiments, the capsule is an enteric capsule.

Further provided is a method of administering a therapeutic agent to a patient, comprising: orally administering to the patient an enteric delivery system in a compacted configuration, the enteric delivery system comprising one or more carrier members comprising a carrier polymer and the therapeutic agent; expanding the enteric delivery system to an expanded configuration; applying, using the expanded enteric delivery system, outwardly directed pressure to the intestinal wall of the small intestine of the patient; and releasing the therapeutic agent from enteric delivery system to transport the therapeutic agent across the enteric mucosa of the small intestine.

In some embodiments of the method, the enteric delivery system is expanded within the small intestine.

In some embodiments of the method, the enteric delivery system expands in the duodenum of the patient.

In some embodiments of the method, the enteric delivery system is expanded within the stomach of the patient and passes through the pylorus of the patient into the small intestine without substantial release of the therapeutic agent until the system enters the small intestine.

In some embodiments of the method, at least a portion of the system loses structural integrity after a period of time within the small intestine to release the outwardly directed pressure. In some embodiments, the outwardly directed pressure is released after about 1 to about 72 hours after the system enters the small intestine. In some embodiments, release of the outwardly directed pressure allows the enteric delivery system to pass through the small intestine.

In some embodiments of the method, the therapeutic agent is a polypeptide or a polynucleotide. In some embodiments, the therapeutic agent is a polypeptide comprising 10 or more amino acids. In some embodiments, the therapeutic agent is a polynucleotide comprising 10 or more nucleotides.

In some embodiments of the method, the enteric delivery system is any one of the above-described systems.

In some embodiments of the method, the patient is a human.

### BRIEF DESCRIPTION OF THE DRAWINGS

FIG. 1 illustrates an exemplary toroidal enteric delivery system.

FIG. 2 shows the toroidal enteric delivery system with a continuous carrier member formed from a tube.

FIG. 3 shows the toroidal enteric delivery system within the lumen of the small intestine.

FIG. 4 shows the toroidal enteric delivery system within the lumen of the small intestine, where the outer diameter of the toroid is larger than the inner dimeter of the lumen of the small intestine.

FIG. 5 illustrates an embodiment of a toroidal enteric delivery system with a single carrier member, with the ends of the carrier member joined together by a linker.

FIG. 6 illustrates an example of a toroidal enteric delivery system with eight carrier members joined end to end by eight linkers, with each linker joining two ends of different carrier members.

FIG. 7 illustrates a ring-shaped enteric delivery system with a continuous carrier member.

FIG. 8 shows the teardrop shaped cross-section of the carrier member of the system shown in FIG. 7.

FIG. 9 illustrates an enteric delivery system with a coating containing the therapeutic agent coated on an outer surface of the carrier member.

FIG. 10 shows a side view of the enteric delivery system illustrated in FIG. 9, with a coating containing the therapeutic agent on the outer surface of the carrier member.

FIG. 11 shows the use of a blade to cut a spiral of carrier polymer to form the carrier member of a ring-shaped system.

FIG. 12 illustrates the ring-shaped enteric delivery system formed as illustrated in FIG. 11.

FIG. 13 illustrates one embodiment of an enteric delivery system in a stellate design in an expanded configuration, which includes an elastomeric central member, and six arms radiating from the central member.

FIG. 14 illustrates a compacted configuration of the enteric delivery system illustrated in FIG. 13.

FIG. 15 illustrates an embodiment of the enteric delivery system with a stellate design, wherein the therapeutic drug is disposed in a coating at the distal tip of the arms, which are attached to the central member through linkers.

FIG. 16 illustrates the enteric delivery system illustrated in FIG. 15, but in the expanded configuration and within the lumen of the small intestine.

FIG. 17 illustrates a toroidal enteric delivery system in a capsule.

FIG. 18 illustrates a toroidal enteric delivery system which is folded in two to further compact the system, in a capsule.

FIG. 19 illustrates a stellate enteric delivery system in a compacted configuration in a capsule.

FIG. 20 shows memantine bioanalysis in plasma collected from dogs that were administered an enteric delivery system. The results showed good exposure from the small intestine with a Tₘₐₓ at 8 hours and sustained release of memantine was measurable for 7 days.

### DETAILED DESCRIPTION OF THE INVENTION

Described herein are enteric delivery systems, which can be useful for delivering therapeutic agents, and in particular biological therapeutic agents such as polypeptides and polynucleotides, to a subject by oral administration. The orally ingested system travels through the stomach and into the small intestine, where it maintains contact with the intestinal wall of the small intestine (preferably, the duodenum) by applying an outward pressure to the intestinal wall. The system expands from a compacted configuration to an expanded configuration, and may expand within the small intestine, or expand within the stomach and travel through the pylorus into the small intestine without substantial release of the therapeutic agent until reaching the small intestine (for example, by including an enteric coating on the system that dissolves only in the small intestine). The sustained contact and outwardly applied pressure allows the therapeutic agents of the enteric delivery system to be absorbed through the enteric mucosa. The enteric delivery system include can include enteric components (such as carrier members, elastomeric central members, or linkers), which are configured to lose structural integrity in the small intestine (for example, on the order of about 1 hour to about 72 hours). Once the system loses its structural integrity, the outwardly directed pressure is released, the system will pass through the small intestine through normal transport within the small intestine (i.e., peristalsis). The remaining components of the system travel through the gastrointestinal tract and are egested.

Once in the small intestine, the enteric delivery system maintains contacts the intestinal wall of the small intestine (preferably, the duodenum) by applying an outwardly directed pressure to the intestinal wall. The high local concentration of the therapeutic agent at the inner surface of the intestine promotes diffusion of the therapeutic agent across the intestinal wall. Additionally, the outwardly directed pressure can manipulate the enteric mucosa, which further enhances permeability of the therapeutic agents across the enteric mucosa and into the patient's bloodstream by thinning the mucosal barrier. Although small molecule drugs are frequently absorbed across the intestinal wall, unaided larger therapeutic agents, such as peptide, proteins, and nucleic acids cannot effectively be absorbed from the small intestine. By manipulating the enteric mucosa using the enteric delivery system described herein, and by maintaining contact between the intestinal wall and the therapeutic drug containing components of the enteric delivery system, at least a portion of the therapeutic drug is transported across the enteric mucosa of the small intestine, thus increasing bioavailability of the small intestine.

The system for enteric delivery of the therapeutic drug includes one or more carrier members comprising a carrier polymer and a therapeutic agent, and the system is configurable in a compacted configuration and an expanded configuration, wherein the system is configured to (1) expand from the compacted configuration to the expanded configuration within the small intestine, or (2) expand from the compacted configuration to the expanded configuration within the stomach and pass through the pylorus without substantial release of the therapeutic agent until reaching the small intestine. Additionally, the system is sized to maintain contact with the intestinal wall of the small intestine by applying an outwardly directed pressure to the intestinal wall and transport at least a portion of the therapeutic agent across the enteric mucosa of the small intestine; and at least a portion of the system loses structural integrity after a period of time within the small intestine to release the outwardly directed pressure.

In one example, the enteric delivery system includes one or more carrier members comprising a carrier polymer and a therapeutic agent arranged in a ring shape. The ring shape may be, for example, toroidal, elliptical, or teardrop-shaped. In some embodiments, the ring shape is formed form a single, continuous carrier member without any joints or welds. In some embodiments the enteric delivery system includes one or more carrier members comprising the therapeutic members, and one or more linkers that join the one or more carrier members to form a ring shape. The carrier members and/or the linkers can include or can be formed of an enteric material, which is configured to lose structural integrity (for example, by degradation, dissolution, or softening) after a period of time within the small intestine.

In another example, the enteric delivery system includes an elastomeric central member attached to a plurality of elongated carrier members that radiate outwardly from the central member. The elongated carrier members include a carrier polymer and the therapeutic agent. Optionally, the elastomeric central member is joined to the carrier members through one or more linker. In some embodiments, the central member, the carrier members, and/or the linkers are formed of an enteric material (such as a polymer) that is configured to lose structural integrity (for example, by degradation, dissolution, or softening) after a period of time within the small intestine.

An enteric delivery system can be used to administer a therapeutic agent to a patient. For example, described herein is a method of administering a therapeutic agent to a patient, comprising orally administering to the patient an enteric delivery system in a compacted configuration, the enteric delivery system comprising one or more carrier members comprising a carrier polymer and the therapeutic agent; expanding the enteric delivery system to an expanded configuration applying, using the expanded enteric delivery system, outwardly directed pressure to the intestinal wall of the small intestine of the patient; and releasing the therapeutic agent from the enteric delivery system to transport the therapeutic agent across the enteric mucosa of the small intestine. Further details of this method are provided herein.

### Definitions

As used herein, the singular forms “a,” “an,” and “the” include the plural references unless the context clearly dictates otherwise.

Reference to “about” a value or parameter herein includes (and describes) variations that are directed to that value or parameter per se, as well as values or parameters that are reasonably close to the value or parameter as specified. For example, description referring to “about X” includes description of “X” as well as those values that are reasonably close to X. If a range is indicated, such as “about X to Y,” it is understood that both the values specified by the endpoints are included, and that values close to each endpoint or both endpoints are included for each endpoint or both endpoints.

The term “antibody” refers to a polypeptide or a set of interacting polypeptides that specifically bind to an antigen, and includes, but is not limited to a monoclonal antibody, polyclonal, a chimeric antibody, a CDR-grafted antibody, a humanized antibody, a Fab, a Fab′, a F(ab′)₂, a Fv, a disulfide linked Fv, a scFv, a single domain antibody (dAb), a diabody, a multispecific antibody, a dual specific antibody, an anti-idiotypic antibody, a bispecific antibody, a functionally active epitope-binding fragment thereof, bifunctional hybrid antibodies, a single chain antibody, and a Fc-containing polypeptide, such as an immunoadhesion. In some embodiments, the antibody may be of any heavy chain isotype (e.g., IgG, IgA, IgM, IgE, or IgD). In some embodiments, the antibody may be of any light chain isotype (e.g., kappa or gamma). The antibody may be non-human (e.g., from mouse, goat, or any other animal), fully human, humanized, or chimeric.

“Biocompatible,” when used to describe a material or system, indicates that the material or system does not provoke an adverse reaction, or causes only minimal, tolerable adverse reactions, when in contact with an organism, such as a human. In the context of the enteric delivery systems, biocompatibility is assessed in the environment of the gastrointestinal tract.

A “carrier polymer” is a polymer suitable for blending with an agent, or a polymer suitable as substrate that can be coated with a coating that contains an agent, for use in the systems described herein.

An “excipient” is any substance added to a formulation of an agent that is not the agent itself. Excipients include, but are not limited to, binders, coatings, diluents, disintegrants, emulsifiers, flavorings, glidants, lubricants, and preservatives. The specific category of dispersant falls within the more general category of excipient.

An “elastic polymer,” “elastomeric polymer,” or “elastomer” is a polymer that is capable of being deformed by an applied force from its original shape for a period of time, and which then substantially returns to its original shape once the applied force is removed.

An “enteric polymer” is a polymer that is generally resistant to acidic pH levels of the stomach, but dissolves at higher pH levels found in the duodenum.

“M_{w}” refers to weight-average molecular weight of a polymer.

A “patient,” “individual,” or “subject” refers to a mammal, preferably a human or a domestic animal such as a dog or cat. In a most preferred embodiment, a patient, individual, or subject is a human.

Reference to a “substantial” amount refers to 95% or more. For example, reference to “release of substantially all” of a therapeutic compound refers to release of 95% or more of the therapeutic drug. In another example, reference to “without substantial release” of a therapeutic drug refers to release of less than 5% of the therapeutic drug.

“Therapeutic use” of the systems disclosed herein is defined as using one or more of the systems disclosed herein to treat a disease or disorder, as defined above. A “therapeutically effective amount” of a therapeutic agent, such as a drug, is an amount of the agent, which, when administered to a patient, is sufficient to reduce or eliminate either a disease or disorder or one or more symptoms of a disease or disorder, or to retard the progression of a disease or disorder or of one or more symptoms of a disease or disorder, or to reduce the severity of a disease or disorder or of one or more symptoms of a disease or disorder. A therapeutically effective amount can be administered to a patient as a single dose, or can be divided and administered as multiple doses.

“Treating” a disease or disorder with the systems and methods disclosed herein is defined as administering one or more of the systems disclosed herein to a patient in need thereof, with or without additional agents, in order to reduce or eliminate either the disease or disorder, or one or more symptoms of the disease or disorder, or to retard the progression of the disease or disorder or of one or more symptoms of the disease or disorder, or to reduce the severity of the disease or disorder or of one or more symptoms of the disease or disorder. “Suppression” of a disease or disorder with the systems and methods disclosed herein is defined as administering one or more of the systems disclosed herein to a patient in need thereof, with or without additional agents, in order to inhibit the clinical manifestation of the disease or disorder, or to inhibit the manifestation of adverse symptoms of the disease or disorder. The distinction between treatment and suppression is that treatment occurs after adverse symptoms of the disease or disorder are manifest in a patient, while suppression occurs before adverse symptoms of the disease or disorder are manifest in a patient. Suppression may be partial, substantially total, or total. Because some diseases or disorders are inherited, genetic screening can be used to identify patients at risk of the disease or disorder. The systems and methods of the present disclosure can then be used to treat asymptomatic patients at risk of developing the clinical symptoms of the disease or disorder, in order to suppress the appearance of any adverse symptoms.

It is understood that aspects and variations of the invention described herein include “consisting” and/or “consisting essentially of” aspects and variations.

When a range of values is provided, it is to be understood that each intervening value between the upper and lower limit of that range, and any other stated or intervening value in that states range, is encompassed within the scope of the present disclosure. Where the stated range includes upper or lower limits, ranges excluding either of those included limits are also included in the present disclosure.

Unless otherwise specified, percentages of ingredients in compositions are expressed as weight percent, or weight/weight percent. It is understood that reference to relative weight percentages in a composition assumes that the combined total weight percentages of all components in the composition add up to 100. It is further understood that relative weight percentages of one or more components may be adjusted upwards or downwards such that the weight percent of the components in the composition combine to a total of 100, provided that the weight percent of any particular component does not fall outside the limits of the range specified for that component.

The section headings used herein are for organization purposes only and are not to be construed as limiting the subject matter described. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

The disclosures of all publications, patents, and patent applications referred to herein are each hereby incorporated by reference in their entireties. To the extent that any reference incorporated by reference conflicts with the instant disclosure, the instant disclosure shall control.

### Enteric Delivery System Overview

The enteric delivery system includes one or more carrier members that include a carrier polymer and a therapeutic agent. The therapeutic agent can be included in a coating that coats the core of the carrier members, or can be loaded into the carrier members. The system is configurable in a compacted configuration (i.e., a small profile) and an expanded configuration (i.e., a large profile). The compacted configuration allows the system to be readily ingested by a patient, and the system expands after being administered. The system is sized such that, once in the expanded configuration and within the small intestine, the system maintains contact with the intestinal wall of the small intestine by applying an outwardly directed pressure to the intestinal wall. The outwardly directed pressure further allows transportation of at least a portion of the therapeutic agent across the enteric mucosa of the small intestine. Additionally, the system is configured such that at least a portion of the system loses structural integrity after a period of time (such as between about 1 hour and about 72 hours) within the small intestine, which releases the outwardly directed pressure.

To allow passage of chyme in the small intestine with the expanded enteric delivery system deployed in the small intestine, the system can include one or more openings (for example, a central opening in a ring structure, or one or more opening in a central member of a stellate structure). The enteric delivery system can be, but need not be, statically positioned within the small intestine. For example, the outwardly directed pressure applied by the system to the intestinal wall may slow or stop movement of the system in the small intestine. As further discussed herein, at least a portion of the system (such as the carrier members, the linkers, and/or the central members, if present) may be designed to lose structural integrity after a period of time within the small intestine (such as about 1 hour to about 72 hours, for example about 1 hour to about 2 hours, about 2 hours to about 4 hours, about 4 hours to about 6 hours, about 6 hours to about 8 hours, about 8 hours to about 12 hours, about 12 hours to about 24 hours, about 24 hours to about 36 hours, about 36 hours to about 48 hours, or about 48 hours to about 72 hours), which releases the outwardly directed pressure. When the system loses structural integrity and the outwardly directed pressure is released, the system, or the remaining portion of the system (for example, components that were not degraded or eroded), can be passed through the small intestine, for example at the rate of ordinary passage within the lumen.

The system is configured to expand from the compacted configuration, which is sized for oral administration, to the expanded configuration. The enteric delivery system can be packaged in the compacted configuration and, when released from the packaging, expand into the expanded configuration. For example, the system can be encapsulated in a capsule and, once released from the capsule, the system expands into the expanded configuration. In some embodiments, the capsule is an enteric capsule. The enteric capsule allows the enteric delivery system to pass through the stomach, where the enteric material of the enteric capsule is maintained due to the low pH of the gastric environment, and into the small intestine. Once in the small intestine, the enteric delivery system expands from the compacted configuration to the expand configuration.

In certain embodiments, the enteric delivery system expands into the expanded configuration within the stomach rather than the small intestine. The enteric delivery system can expand in the stomach and pass through the pylorus to enter the small intestine without substantial release of the therapeutic agent until reaching the small intestine. Optionally, an enteric coating (which may have a thickness, for example, of about 2 μm to about 300 μm thick (such as about 2 μm to about 5 μm, about 5 μm to about 10 μm thick, about 10 μm to about 20 μm thick, about 20 μm to about 30 μm thick, about 30 μm to about 50 μm thick, about 50 μm to about 100 μm, about 100 μm to about 150 μm, about 150 μm to about 200 μm, about 200 μm to about 250 μm, or about 250 μm to about 300 μm) coats the enteric delivery system to protect the system from the gastric environment. In some embodiments, the enteric coating coats a coating containing the therapeutic agent, and in some embodiments, the enteric coating includes the therapeutic agent. The enteric coating, if present, surrounds or includes within its matrix the therapeutic agent (that is, it coats the carrier member or the coating containing the therapeutic agent if present, or contains within its protective composition the therapeutic agent) to prevent release of the therapeutic agent within the stomach. The enteric coating can dissolve or degrade in the small intestine, which allows the therapeutic agent to be released from the enteric delivery system.

The expanded enteric delivery system is sized to maintain contact with the intestinal wall of the small intestine (such as the duodenum) by applying an outwardly directed pressure to the intestinal wall and transport at least a portion of the therapeutic agent across the enteric mucosa. In some embodiments, the diameter of the expanded enteric delivery system is at least the diameter of the small intestine or duodenum (such as about 1 times to about 2 times the diameter of the small intestine, for example about 1 times to about 1.1 times, about 1.1 times to about 1.2 times, about 1.2 times to about 1.3 times, about 1.3 times to about 1.4 times, about 1.4 times to about 1.5 times, about 1.5 times to about 1.6 times, about 1.6 times to about 1.7 times, about 1.7 times to about 1.8 times, about 1.8 times to about 1.9 times, or about 1.9 times to about 2 times the diameter of the small intestine or duodenum). In some embodiments, the circumference of the expanded enteric delivery system is at least the inner circumference of the small intestine or duodenum (such as about 1 times to about 2 times the diameter of the small intestine, for example about 1 times to about 1.1 times, about 1.1 times to about 1.2 times, about 1.2 times to about 1.3 times, about 1.3 times to about 1.4 times, about 1.4 times to about 1.5 times, about 1.5 times to about 1.6 times, about 1.6 times to about 1.7 times, about 1.7 times to about 1.8 times, about 1.8 times to about 1.9 times, or about 1.9 times to about 2 times the diameter of the small intestine or duodenum). An enteric delivery system larger than the small intestine or duodenum may rest in the intestine as an elongated or partially compressed (although still expanded compared to the compacted configuration) structure, that sits in a plane oblique to the axis of the intestinal lumen, particularly if the enteric delivery system is a ring-shaped system.

The carrier members include a carrier polymer, which may be a pliable or elastomeric polymer. In some embodiments, the carrier polymer is an enteric polymer, which is configured to lose structural integrity after a period of time within the small intestine. The carrier members are generally elongated, and may be configured to obtain the desired shape of the enteric delivery system. In an expanded configuration of the enteric delivery system, the carrier may be straight (such as in the stellate design) or may be curved (such as in the ring shape design).

The carrier members can have a solid core or a hollow core (i.e., tubular). The outwardly pressure applied by the enteric delivery system to the intestinal wall can depend on the thickness of the solid carrier member or the thickness of the tubular wall of a tubular carrier member, as well as the material of the carrier members.

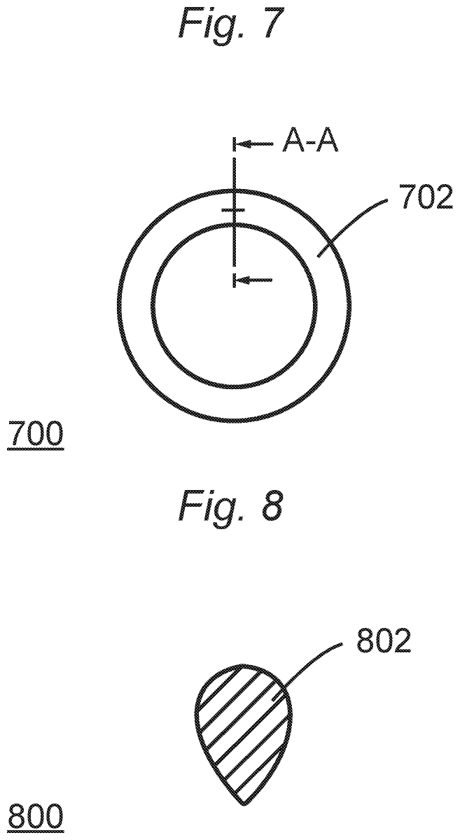

The cross-section of the carrier members may be round (e.g., circular or ellipsoidal), semi-circular, crescent, polygonal (e.g., triangular, square, rectangular, pentagonal, hexagonal, etc.), teardrop shaped, eye shaped, or any other suitable shape. FIG. 7, for example illustrates a ring-shaped enteric delivery system 700 with a continuous carrier member 702. The cross-section of the carrier member along A-A is illustrated in FIG. 8. As shown in FIG. 8, the cross-section of the carrier member 802 is teardrop shaped.

### Ring-Shaped Enteric Delivery System

In some embodiments of the enteric delivery system, the system is ring-shaped. The ring shape refers to the looped design of the structure, with the one or carrier members being attached end-to-end to form a continuous loop (which may be directly joined, for example by welding, or may be linked by one or more linkers). The ring shape also refers embodiments that include a single, continuous carrier member. Exemplary ring shapes include teardrop shaped, ellipsoidal, toroidal, and eye shaped structures.

The ring shaped may be formed by joining ends of linear, but flexible, carrier members end-to-end, for example using an a linker (such as an adhesive polymer, which may be enteric and configured to lose structural integrity, soften, degrade, erode, or break after a period of time within the small intestine) or by welding the ends of the carrier members together.

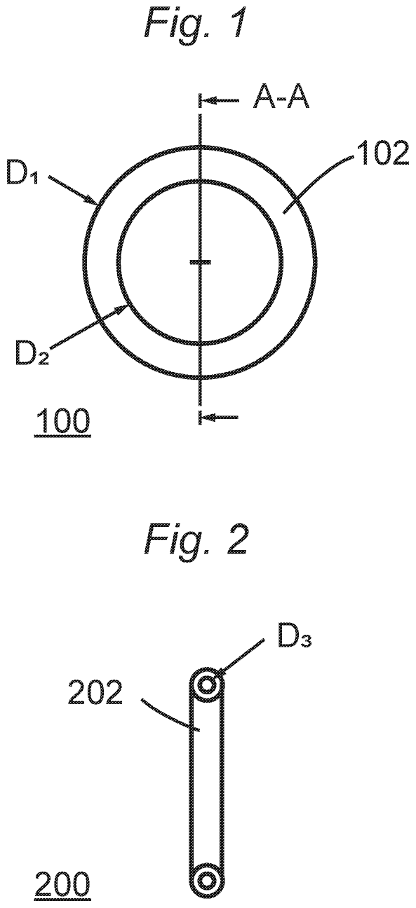

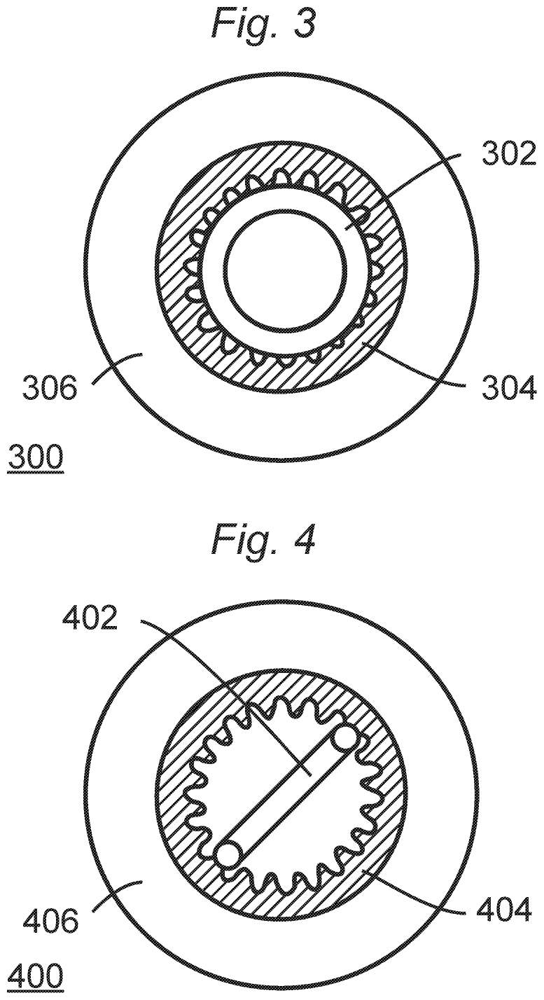

FIG. 1 illustrates an exemplary toroidal enteric delivery system 100. The Enteric delivery system includes a continuous carrier member 102. The system has an outer diameter D₁ and an inner diameter D₂, and the thickness of the carrier member is the difference between the outer diameter and the inner diameter. The ring-shape of the system includes an open center defined by the inner diameter D₂. The open center allows for the passage of chyme through the open center when the enteric delivery system is in the small intestine. A cross-section along line A-A in FIG. 1 is illustrated in FIG. 2. FIG. 2 shows the toroidal enteric delivery system 200 with a continuous carrier member 202 formed from a tube. The carrier member 202 therefore has a hollow core, which is defined by the core diameter D₃. The outer diameter D₁ is about the same or larger than the inner diameter of the small intestine (or duodenum). FIG. 3 shows the toroidal enteric delivery system 302 within the lumen of the small intestine, which includes a mucosal layer 304 and a muscle layer 306. In the embodiment illustrated in FIG. 3, the enteric delivery system is positioned perpendicular to the intestinal wall, with the central opening of the system parallel to the axis of the small intestine lumen. The outer dimeter D₁ may also be larger than the inner diameter of the small intestine, as shown in FIG. 4. In FIG. 4, the toroidal enteric delivery system is positioned within the small intestine, which includes the mucosal layer 404 and a muscle layer 406, such that the ring shape is elongated along the axis of the intestinal lumen. In this configuration the central opening of the ring shape may be perpendicular or oblique to the axis of the lumen. In both the configuration illustrated in FIG. 3 and the configuration illustrated in FIG. 4, the outer surface of the ring-shaped enteric delivery system maintains contact with the intestinal wall of the small intestine by applying an outwardly directed pressure to the intestinal wall. Therapeutic agent loaded into a carrier polymer of the carrier member or in a coating of the carrier member can diffuse from the carrier member to the mucosal layer (304 or 404), which allows the system to transport at least a portion of the therapeutic agent across the enteric mucosa of the small intestine.

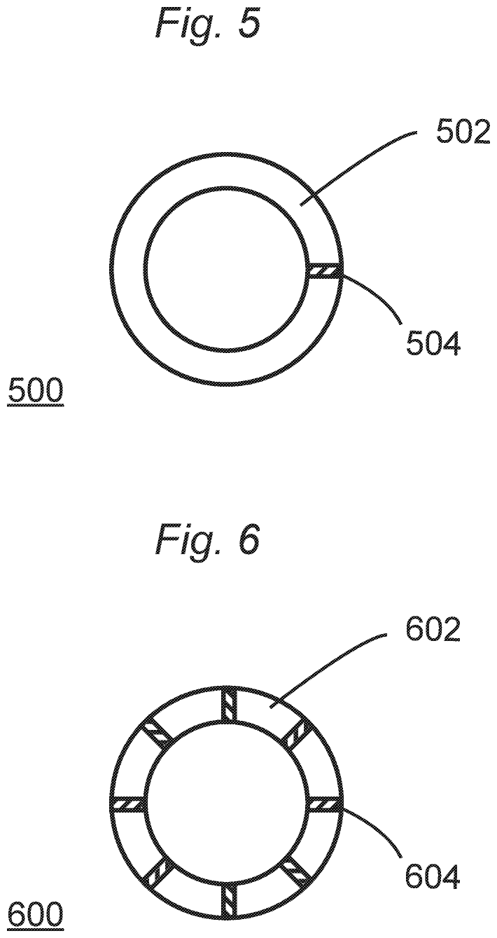

The ring-shaped enteric delivery system can include a single continuous carrier member, or can include one or more carrier members joined end-to-end to form a ring shape. In some embodiments, the ends of the one or more carrier members are joined through one or more linkers. The width of the linker can be about the same as the width of the carrier members, which creates a smooth surface of the system. The carrier polymer of the carrier member and/or the linker can include an enteric material, which is configured to lose structural integrity after a period of time within the small intestine. FIG. 5 illustrates an embodiment of a toroidal enteric delivery system with a single carrier member 502, with the ends of the carrier member 502 joined together by a linker 504. FIG. 6 illustrates an example of a toroidal enteric delivery system with eight carrier members 602 joined end to end by eight linkers 604, with each linker joining two ends of different carrier members 602. The ring shaped enteric delivery system may include any suitable number of carrier members and linkers, such as 1, 2, 3, 4, 5, 6, 7, 8, 9, 10, 11, 12, 13, 14, 15, 16, 17, 18, 19, 20 or more carrier members and 1, 2, 3, 4, 5, 6, 7, 8, 9, 10, 11, 12, 13, 14, 15, 16, 17, 18, 19, 20 or more linkers.

In some ring-shaped enteric delivery system designs, the one or more carrier members are joined by welding the ends of the carrier members, by joining the ends of the carrier members through a linker, or a mixture thereof (i.e., some carrier members are joined by welding and some carrier members are joined through a linker). Ring shaped-enteric delivery systems with a single continuous carrier member may be formed by slicing a large tube (which may be formed through extrusion) to form the ring shapes; this process does not require welding or joining of ends through a linker.

Additional ring-shape enteric delivery systems can be in the form of a teardrop or eye shape. These exemplary configurations may have sharp angles within the ring structure. To form these structures, the carrier members may have angled ends, which can reduce the mechanical stress on the weld or linker joining the ends of the carrier members.

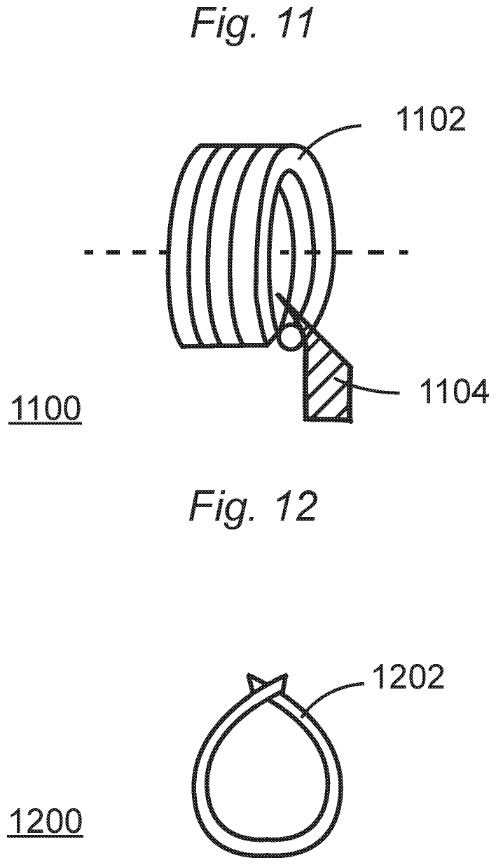

FIG. 12 illustrates another example of a ring-shaped enteric delivery system. The enteric delivery system illustrated in FIG. 12 includes a single carrier member 1202 that is cut from a spiral of carrier polymer 1102 using a blade 1104, as shown in FIG. 11. The cut ends of the carrier member may be cut perpendicularly, or may be cut at an angle. The ends of the cut carrier material can be joined together, for example by welding or depositing a linker between the two cut ends.

The ring-shaped systems optionally include bends or hinges (which may be elastomeric linkers), which can be useful for guiding folding of the system into a compacted configuration, for example for loading into a capsule or other packaging.

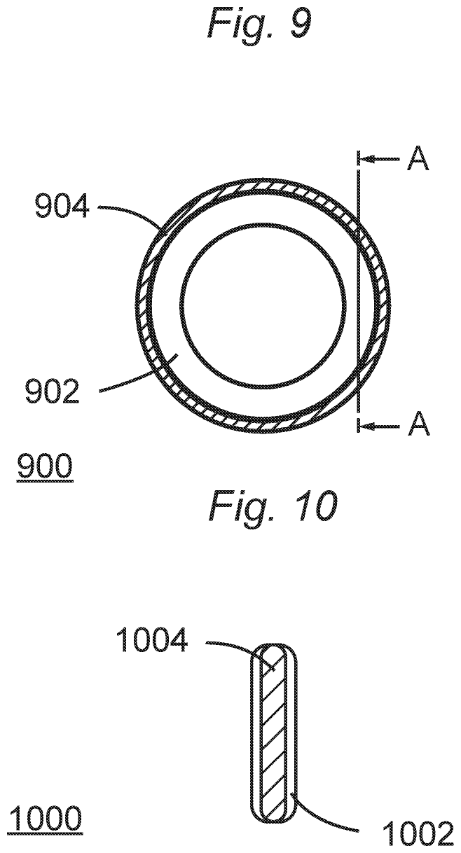

The therapeutic agent of the ring-shaped system can be disposed within the carrier members (for example, loaded into the carrier polymer of the carrier member), or can be coated on the carrier member (for example, coated on the carrier polymer, which may or may not include one or more intervening layers between the coating comprising the therapeutic agent and the carrier polymer). The coating containing the therapeutic agent may be coated on the entire carrier member, or a portion or surface of the carrier member, for example an outer portion or surface of the carrier member or ring-shaped system. FIG. 9 illustrates an enteric delivery system 900 with a coating 904 containing the therapeutic agent coated on an outer surface of the carrier member 902. A side perspective view of the enteric delivery system 900 illustrated in FIG. 9 along A-A is illustrated in FIG. 10, which shows the enteric delivery system 1000 with a coating 1004 containing the therapeutic agent on the outer surface of the carrier member 1002.

The portion of the system that includes the therapeutic agent (e.g., a coating containing the therapeutic agent on the carrier members, or the carrier polymer containing the therapeutic agent) maintains contact with the intestinal wall and applies an outwardly directed pressure to the intestinal wall. The outwardly directed pressure, along with the maintained contact of the portion of the system with the therapeutic agent, allows at least a portion of the therapeutic agent to be transported across the intestinal wall.

After a period of time within the small intestine, at least a portion of the system (such as one or more of the carrier members or one or more of the linker) loses structural integrity (for example, by degradation, dissolution, erosion, or softening). The loss of structural integrity results in a release of the outwardly directed pressure. In some embodiments, the component of the system loses structural integrity after being in the small intestine for about 1 hour to about 72 hours (for example about 1 hour to about 2 hours, about 2 hours to about 4 hours, about 4 hours to about 6 hours, about 6 hours to about 8 hours, about 8 hours to about 12 hours, about 12 hours to about 24 hours, about 24 hours to about 36 hours, about 36 hours to about 48 hours, or about 48 hours to about 72 hours).

### Stellate-Shaped Enteric Delivery System

In some embodiments of the enteric delivery system, the system is designed in a stellate shape. The stellate shape includes a central member with a plurality of arms radiating outwardly from the central member. The central member is generally elastomeric, which allows the system to be compacted by positioning the distal ends (relative to the central member) of the arms adjacent to each other. In the expanded configuration, the arms and the central member lie in plane with each other such that the system is flat. The arms include at least one carrier member, but can include two, three, four or more carrier members joined together end-to-end by one or more linkers. When an arm contains a plurality of carrier members, the carrier members may be of the same or different materials. The arms may be directly attached to the central member, or may be attached through one or more linkers, which optionally include a polymer configured to lose structural integrity after a period of time in the small intestine.

Depending on the size and flexibility of the enteric delivery system, the contact between the system and the intestinal wall can be along the length of the arms or the distal ends of the arms. Contact is maintained with the intestinal wall of the small intestine by applying an outwardly directed pressure to the intestinal wall. The therapeutic agent may be distributed along the length of the arms, or may be preferentially included in (for example, within the carrier polymer) or on (for example, in a coating of the carrier members) the distal ends of the arms.

The stellate structure can include any suitable number of arms, such as 3, 4, 5, 6, 7, 8, 9, 10, 11, 12, 13, 14, or 15 or more arms. The arms may be stiff or soft, or can include a stiff portion and a soft portion. Stiff materials may facilitate the application of outwardly directed pressure to the intestinal wall, while soft materials may achieve greater contact area with the intestinal wall. In some embodiments the arms include a first portion or first carrier member proximal to the central member that is stiff, and a second portion or second carrier member distal from the central member that is soft. In some embodiments the arms include a first portion or first carrier member proximal to the central member that is soft, and a second portion or second carrier member distal from the central member that is stiff. Stiffness is generally measured as a Young's modulus, and components (such as the arms, or a portion of the arms) can have a stiffness between about 1 MPa and about 1 GPa (for example about 1 MPa to about 5 MPa, about 5 MPa to about 10 MPa, about 10 MPa to about 20 MPa, about 20 MPa to about 50 MPa, about 50 MPa to about 100 MPa, about 100 MPa to about 250 MPa, about 250 MPa to about 500 MPa, about 500 MPa to about 750 MPa, or about 750 MPa to about 1 GPa).

The therapeutic agent in the stellate-shape enteric delivery system can be on (e.g., coated on) or in (e.g., loaded into a carrier polymer) of the carrier members. In some embodiments, the therapeutic drug is evenly distributed on or in the carrier members, and in some embodiments the therapeutic drug is preferentially disposed on or within the distal ends of the arms, relative to the central member. For example, in some embodiments, about 80% or more, about 85% or more, about 90% or more, about 95% or more, about 97% or more, about 98% or more, about 991% or more or about 99.5% or more of the therapeutic agent is in or on the distal 5%, distal 10%, distal 15%, distal 20%, distal 25%, distal 30%, distal 35%, or distal 40% portion of the arms.

The components of the enteric delivery system (central member, carrier member and/or linkers) can be configured to lose structural integrity after a period of time in the small intestine (such as about 1 hour to about 72 hours, for example about 1 hour to about 2 hours, about 2 hours to about 4 hours, about 4 hours to about 6 hours, about 6 hours to about 8 hours, about 8 hours to about 12 hours, about 12 hours to about 24 hours, about 24 hours to about 36 hours, about 36 hours to about 48 hours, or about 48 hours to about 72 hours). For example one or more of the components can include a material that erodes, degrades, dissolves or softens within the intestine, such as an enteric material or hydrogel.

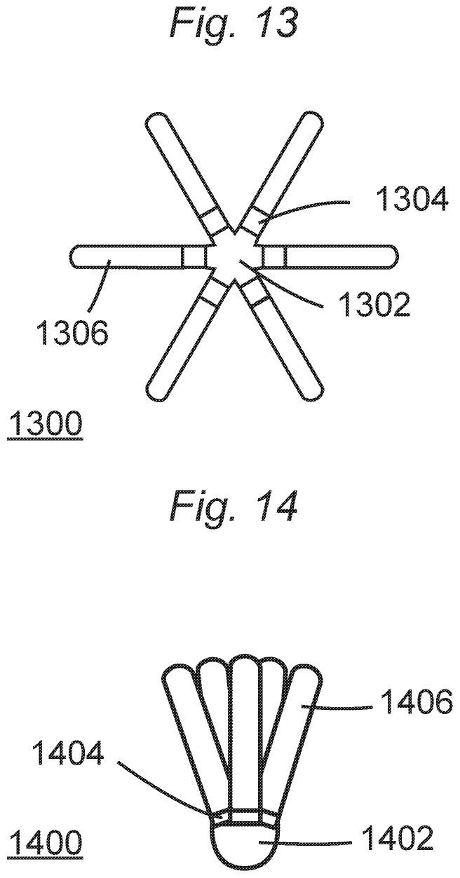

FIG. 13 illustrates one embodiment of an enteric delivery system in a stellate design in an expanded configuration, which includes an elastomeric central member 1302, and six arms 1306 radiating from the central member 1302. Each arm 1306 in the illustrated embodiment is attached to the central member by a linker 1304. The arms are the carrier members of the device, and include the therapeutic agent within or coated on the carrier members. FIG. 14 illustrates a compacted configuration of the enteric delivery system illustrated in FIG. 13. The central member 1402 is elastomeric, which allows for mobility of the arms 1406 of the device. The arms 1406 are connected to the elastic central member 1402 through linkers 1404. In the compacted configuration, the arms 1406 are clustered together and the central member 1402 is stretched. However, release of the enteric delivery system (for example, release from a capsule) relaxes the elastomeric central member 1402, and the arms 1406 reposition outwardly, as shown in FIG. 13.

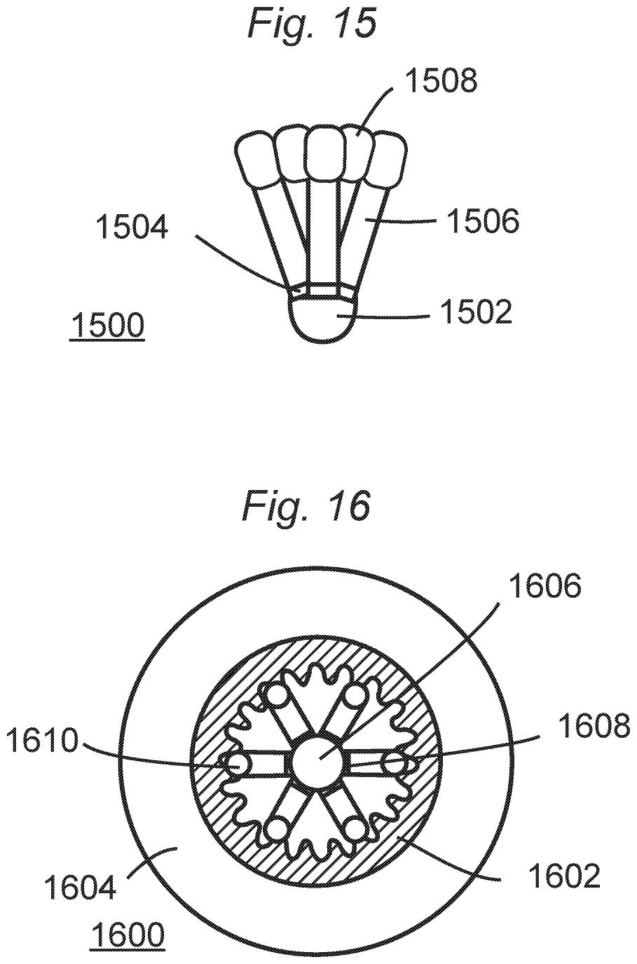

FIG. 15 illustrates an embodiment of the enteric delivery system 1500 with a stellate design, wherein the therapeutic drug is disposed in a coating 1508 at the distal tip of the arms 1506, which are attached to the central member 1502 through linkers. The enteric delivery system 1500 is illustrated in FIG. 15 in the compact configuration. FIG. 16 illustrates the enteric delivery system illustrated in FIG. 15, but in the expanded configuration and within the lumen of the small intestine. The elastomeric central core 1606 of the system is relaxed, which allows the arms of the system (which are connected to the central core 1606 vial linkers 1608) to radiate from the central member. The distal ends of the arms are coated with a coating 1610 that includes the therapeutic agent. When the enteric delivery system is within the lumen of the small intestine, the coating 1610 of the system maintains contact with the enteric mucosa 1602 of the intestinal wall, and applies an outwardly directed pressure to the intestinal wall. With the therapeutic agent in the coating 1610, the maintained contact, and the outwardly directed pressure, the therapeutic agent is transported across the enteric mucosa. Surrounding the enteric mucosa 1602 is a muscle layer 1604.

After a period of time within the small intestine, at least a portion of the system (such as one or more of the carrier members, one or more of the linkers, and/or the central member) loses structural integrity (for example, by degradation, dissolution, erosion, or softening). The loss of structural integrity results in a release of the outwardly directed pressure. In some embodiments, the component of the system loses structural integrity after being in the small intestine for about 1 hour to about 72 hours (for example about 1 hour to about 2 hours, about 2 hours to about 4 hours, about 4 hours to about 6 hours, about 6 hours to about 8 hours, about 8 hours to about 12 hours, about 12 hours to about 24 hours, about 24 hours to about 36 hours, about 36 hours to about 48 hours, or about 48 hours to about 72 hours).

### Components of the Enteric Delivery System and Exemplary Materials

One or more of the components of the enteric delivery system, such as the carrier members, the linkers, and/or central members, can include an elastomeric material and/or a material configured to lose structural integrity over a period of time in the small intestine, such as an enteric material. For example, the materials can erode, dissolve, degrade, break, and/or soften (for example, by absorbing water) after a period of time in the small intestine.

The systems described herein are configurable in a compacted configuration, for example to be packaged into a capsule, often for prolonged storage. To ensure reliable expansion of the system, the carrier polymers, linker materials, and central members preferably undergo minimal permanent defamation under prolonged storage in the compacted configuration. Exemplary materials for the system components include elastomeric materials, such as silicone (or silicone rubber) other thermoplastic elastomers.

Some of the materials used in the components of the system described herein are enteric materials or enteric polymers. Enteric materials are configured to resist the acidic gastric environment, but erode, dissolve, degrade, swell, soften, or otherwise lose structural integrity in the higher pH levels of the small intestine. Some of the enteric polymers that can be used in the systems disclosed herein are listed in the Enteric Polymer Table.

Enteric Polymer Table

| Polymer | Dissolution pH |
|---|---|
| Cellulose acetate phthalate | 6.0-6.4 |
| Hydroxypropyl methylcellulose phthalate 50 | 4.8 |
| Hydroxypropyl methylcellulose phthalate 55 | 5.2 |
| Polyvinylacetate phthalate | 5.0 |
| Methacrylic acid-methyl methacrylate copolymer (1:1) | 6.0 |
| Methacrylic acid-methyl methacrylate copolymer (2:1) | 6.5-7.5 |
| Methacrylic acid-ethyl acrylate copolymer (2:1) | 5.5 |
| Shellac | 7.0 |
| Hydroxypropyl methylcellulose acetate succinate | 7.0 |
| Poly (methyl vinyl ether/maleic acid) monoethyl ester | 4.5-5.0 |
| Poly (methyl vinyl ether/maleic acid) n-butyl ester | 5.4 |

Preferably, enteric polymers that dissolve at a pH of no less than about 5 or about 5.5 are used. Poly(methacrylic acid-co-ethyl acrylate) (sold under the trade name EUDRAGIT L 100-55; EUDRAGIT is a registered trademark of Evonik Röhm GmbH, Darmstadt, Germany) and hydroxypropylmethylecllulose acetate succinate (hypromellose acetate succinate or HPMCAS; Ashland, Inc., Covington, Ky., USA) are exemplary enteric polymers. Cellulose acetate phthalate, cellulose acetate succinate, and hydroxypropyl methylcellulose phthalate, are also suitable enteric polymers.

In one embodiment, the enteric polymers used in the system dissolve at a pH above about 4. In some embodiments, the enteric polymers used in the system dissolve at a pH above about 5. In some embodiments, the enteric polymers used in the system dissolve at a pH above about 6. In some embodiments, the enteric polymers used in the system dissolve at a pH above about 7. In some embodiments, the enteric polymers used in the system dissolve at a pH above about 7.5. In some embodiments, the enteric polymers used in the system dissolve at a pH between about 4 and about 5. In some embodiments, the enteric polymers used in the dissolve at a pH between about 4 and about 6. In some embodiments, the enteric polymers used in the dissolve at a pH between about 4 and about 7. In some embodiments, the enteric polymers used in the system dissolve at a pH between about 4 and about 7.5. In some embodiments, the enteric polymers used in the system dissolve at a pH between about 5 and about 6. In some embodiments, the enteric polymers used in the system dissolve at a pH between about 5 and about 7. In some embodiments, the enteric polymers used in the system dissolve at a pH between about 5 and about 7.5. In some embodiments, the enteric polymers used in the system dissolve at a pH between about 6 and about 7. In some embodiments, the enteric polymers used in the system dissolve at a pH between about 6 and about 7.5.

### Carrier Members and Materials for Carrier Members

The carrier members include a carrier polymer, and a therapeutic agent that is loaded into the carrier polymer or coated on the carrier polymer. The carrier polymer may be a homogenous polymer, or may be a blend of two or more polymers. Additionally, the carrier polymer can be blended with one or more excipients (such as a porogen). For example, the carrier polymer can be a blend of a non-erodible polymer and a porogen (e.g., an erodible polymer, an enteric polymer, and/or a swellable hydrogel polymer). The porogens can dissolve, erode, degrade, swell, and/or soften in the small intestine, which causes the carrier member to lose structural integrity even if there is no loss in integrity of the non-erodible polymer of the carrier member. The amount and type of porogen can be selected based on a desired rate of loss of structural integrity of the carrier members.

In some embodiments, the carrier polymer is configured to lose structural integrity over a period of time in the small intestine, for example on the order of about 1 hour to about 72 hours. In some embodiments, the carrier polymer loses structural integrity after being in the small intestine for about 1 hour to about 2 hours, about 2 hours to about 4 hours, about 4 hours to about 6 hours, about 6 hours to about 8 hours, about 8 hours to about 12 hours, about 12 hours to about 24 hours, about 24 hours to about 36 hours, about 36 hours to about 48 hours, or about 48 hours to about 72 hours). Loss of structural integrity of the carrier polymer causes loss of structural integrity of the carrier member, which causes a release of the outwardly applied pressure to the intestinal wall when the system is within the small intestine lumen.

In some embodiments, the carrier polymer is an enteric polymer. In some embodiments, the carrier polymer comprises silicone or a silicone rubber. In some embodiments, the carrier polymer comprises a thermoplastic elastomer. Additional exemplary carrier polymers suitable for use in the systems disclosed herein include, but are not limited to, hydrophilic cellulose derivatives (such as hydroxypropylmethyl cellulose, hydroxypropyl cellulose, hydroxymethyl cellulose, hydroxyethyl cellulose, carboxymethylcellulose, sodium-carboxymethylcellulose), cellulose acetate phthalate, poly(vinyl pyrrolidone), ethylene/vinyl alcohol copolymer, poly(vinyl alcohol), carboxyvinyl polymer (Carbomer), Carbopol® acidic carboxy polymer, polycarbophil, poly(ethyleneoxide) (Polyox WSR), polysaccharides and their derivatives, polyalkylene oxides, polyethylene glycols, chitosan, alginates, pectins, acacia, tragacanth, guar gum, locust bean gum, polyvinylprrolidone, vinylpyrrolidonevinyl acetate copolymer, dextrans, natural gum, agar, agarose, sodium alginate, carrageenan, fucoidan, furcellaran, laminaran, hypnea, eucheuma, gum arabic, gum ghatti, gum karaya, arbinoglactan, amylopectin, gelatin, gellan, hyaluronic acid, pullulan, scleroglucan, xanthan, xyloglucan, maleic anhydride copolymers, ethylenemaleic anhydride copolymer, poly(hydroxyethyl methacrylate), ammoniomethacrylate copolymers (such as Eudragit RL or Eudragit RS), poly(ethylacrylate-methylmethacrylate) (Eudragit NE), Eudragit E (cationic copolymer based on dimethylamino ethyl methylacrylate and neutral methylacrylic acid esters), poly(acrylic acid), polymethacrylates/polyethacrylates such as poly(methacrylic acid), methylmethacrylates, and ethyl acrylates, polylactones such as poly(caprolactone), polyanhydrides such as poly[bis-(p-carboxyphenoxy)-propane anhydride], poly(terephthalic acid anhydride), polypeptides such as polylysine, polyglutamic acid, poly(ortho esters) such as copolymers of DETOSU with diols such as hexane diol, decane diol, cyclohexanedimethanol, ethylene glycol, polyethylene glycol and incorporated herein by reference those poly(ortho) esters described and disclosed in U.S. Pat. No. 4,304,767, starch, in particular pregelatinized starch, and starch-based polymers, carbomer, maltodextrins, amylomaltodextrins, dextrans, poly(2-ethyl-2-oxazoline), poly(ethyleneimine), polyurethane, poly(lactic acid), poly(glycolic acid), poly(lactic-co-glycolic acid) (PLGA), polyhydroxyalkanoates, polyhydroxybutyrate, and copolymers, mixtures, blends and combinations thereof.

In some embodiments, the carrier member comprises a carrier polymer and a porogen. The porogen can be any suitable material that degrades, erodes, dissolves, softens, swells or otherwise loses structural integrity in the small intestine over a period of time. In some embodiments, the porogen is an enteric material, such as an enteric polymer. Examples of porogens include alkali metal salts such as sodium chloride, sodium bromide, potassium chloride, potassium sulfate, potassium phosphate, sodium benzoate, sodium acetate, sodium citrate, potassium nitrate and the like; alkaline earth metal salts such as calcium chloride, calcium nitrate, and the like; and transition metal salts such as ferric chloride, ferrous sulfate, zinc sulfate, cupric chloride, and the like. Additional examples of porogens include saccharides and sugars, such as sucrose, glucose, fructose, mannose, galactose, aldohexose, altrose, talose, lactose, cellulose, monosaccharides, disaccharides, and water soluble polysaccharides. Additional examples of porogens include sorbitol, mannitol, organic aliphatic and aromatic oils, including diols and polyols, as exemplified by polyhydric alcohols, poly(alkylene glycols), polyglycols, alkylene glycols, poly(a,m)alkylenediol esters or alkylene glycols, poly vinylalcohol, poly vinyl pyrrolidone, and water soluble polymeric materials. Further examples of porogens that can be used include Poloxamer; hypromellose (HPMC); Kolliphor RH40; polyvinyl caprolactam; polyvinyl acetate (PVAc); polyethylene glycol (PEG); Soluplus (available from BASF; a copolymer of polyvinyl caprolactam, polyvinyl acetate, and polyethylene glycol); copovidone; Eudragits (E, RS, RL); poly(methyl vinyl ether-alt-maleic anhydride); polyoxyethylene alkyl ethers; polysorbates; polyoxyethylene stearates; polydextrose; polyacrylic acid; alginates; sodium starch glycolate (SSG); crosslinked polyacrylic acid (carbopol); crosslinked PVP (crospovidone); crosslinked cellulose (croscarmellose); calcium silicate; xanthan gum; and gellan gum. Some particularly useful porogens include povidone, copovidone, and polyoxyl castor oil. Porogens can be added to make up between about 1% to about 30% by weight of the carrier member. Porogens can be added to make up about 1% to about 25%, about 1% to about 20%, about 1% to about 15%, about 1% to about 10%, about 1% to about 8%, about 1% to about 5%, about 1% to about 3%, about 5% to about 30%, about 10% to about 30%, about 15% to about 30%, about 20% to about 30%, or about 25% to about 30% by weight of the carrier material.

One or more additional excipients may be included in the carrier member, particularly when the therapeutic agent is disposed within the carrier member. Such additional excipients are discussed with respect to the coating containing the therapeutic agent; however, the excipients can be included in the carrier member, particularly when no coating is present in the system.

### Linkers and Exemplary Materials

Linkers can be included in the enteric delivery system to join carrier members together, or to join carrier members to a central member. The linkers may be more or less prone to losing structural integrity in the small intestine compared to the central member and/or the carrier member(s). In some embodiments, the linkers comprise an enteric polymer and/or a porogen. Exemplary enteric polymers and porogens are identified herein. By way of example, in some embodiments the linker comprises hydroxypropyl methylcellulose (HPMC) or hydroxypropyl methyl cellulose acetate succinate (HPMCAS).

In some embodiments, the linker comprises a plasticizer, such as triacetin, triethyl citrate, tributyl citrate, poloxamers, polyethylene glycol, polypropylene glycol, diethyl phthalate, dibutyl sebacate, glycerin, castor oil, acetyl triethyl citrate, acetyl tributyl citrate, polyethylene glycol monomethyl ether, sorbitol, sorbitan, a sorbitol-sorbitan mixture, or diacetylated monoglycerides.

In some embodiments, the linker is configured to lose structural integrity (for example, by dissolving, eroding, degrading, swelling, softening, or otherwise) over a period of time in the small intestine, for example on the order of about 1 hour to about 72 hours. This feature is particularly useful if, for example, the carrier member and/or the central member are not configured to lose structural integrity over a period of time in the small intestine. In some embodiments, the linker loses structural integrity after being in the small intestine for about 1 hour to about 2 hours, about 2 hours to about 4 hours, about 4 hours to about 6 hours, about 6 hours to about 8 hours, about 8 hours to about 12 hours, about 12 hours to about 24 hours, about 24 hours to about 36 hours, about 36 hours to about 48 hours, or about 48 hours to about 72 hours). Loss of structural integrity of the linker causes loss of structural integrity of the system, which causes a release of the outwardly applied pressure to the intestinal wall when the system is within the small intestine lumen.

### Central Member and Exemplary Materials

The central member of the enteric delivery system, if present (such as in a stellate design) is preferably elastomeric (i.e., includes an elastomer). In some embodiments, the central member comprises an enteric material.

In some embodiments, the central member is configured to lose structural integrity (for example, by dissolving, eroding, degrading, swelling, softening, or otherwise) over a period of time in the small intestine, for example on the order of about 1 hour to about 72 hours. In some embodiments, the central member loses structural integrity after being in the small intestine for about 1 hour to about 2 hours, about 2 hours to about 4 hours, about 4 hours to about 6 hours, about 6 hours to about 8 hours, about 8 hours to about 12 hours, about 12 hours to about 24 hours, about 24 hours to about 36 hours, about 36 hours to about 48 hours, or about 48 hours to about 72 hours). Loss of structural integrity of the central member causes loss of structural integrity of the system, which causes a release of the outwardly applied pressure to the intestinal wall when the system is within the small intestine lumen.

Elastomers enable the enteric delivery system to be compacted, such as by being folded or compressed, into a form suitable for administration to the stomach by swallowing a container or capsule containing the compacted system. Upon dissolution of the capsule in the stomach, the enteric delivery system expands into a shape which prevents passage of the system through the pyloric sphincter of the patient for the desired residence time of the system. Thus, the elastomer must be capable of being stored in a compacted configuration in a capsule for a reasonable shelf life, and of expanding to its original shape, or approximately its original shape, upon release from the capsule. In one embodiment, the elastomer is a silicone elastomer. In one embodiment, the elastomer is formed from a liquid silicone rubber (LSR), such as sold in the Dow Corning QP-1 liquid silicone rubber kit. In one embodiment, the elastomer is crosslinked polycaprolactone. In one embodiment, the elastomer is an enteric polymer, such as those listed in the Enteric Polymer Table. In some embodiments, the coupling polymer(s) used in the system are also elastomers. Elastomers are preferred for use as the central member in the stellate design of the enteric delivery systems.

In one embodiment, both the coupling polymer and elastomer are enteric polymers, which provides for more complete breakage of the system into the carrier polymer-agent pieces if the system enters the intestine, or if the patient drinks a mildly basic solution in order to induce passage of the system.

Examples of elastomers which can be used include silicones, such as those formed using Dow Corning QP-1 kits; urethane-cross-linked polycaprolactones; poly(acryloyl 6-aminocaproic acid) (PA6ACA); poly(methacrylic acid-co-ethyl acrylate) (EUDRAGIT L 100-55); and mixtures of poly(acryloyl 6-aminocaproic acid) (PA6ACA) and poly(methacrylic acid-co-ethyl acrylate) (EUDRAGIT L 100-55).

Flexible coupling polymers. i.e., elastomeric coupling polymers or elastomers, are used as the central member in the stellate design of the enteric delivery systems. A particularly preferred elastomer for use as the central elastomer of the stellate or star configuration is silicone rubber. Liquid silicone rubber (LSR) can be molded easily and cured into a desired shape. The Dow Corning QP-1 series, comprising cross-linked dimethyl and methyl-vinyl siloxane copolymers and reinforcing silica, are examples of such silicone rubber polymers (see, for example, the Web site www.dowcoming.com/DataFiles/090276fe8018ed07.pdf). Non-segmented elongate members or elongate members comprising segments of carrier polymer-agent components can then be attached to the central silicone rubber elastomer. Another elastomer which can be used as the central elastomer in the stellate design is crosslinked polycaprolactone.

### Therapeutic Agents, Coatings. and Excipients

The therapeutic agent can be included in the system either within the carrier member (i.e., mixed with the carrier polymer) or on the carrier member (i.e., a coating covering the carrier member or a portion of the carrier member). Excipients can be included with the therapeutic agent, for example in the coating or combined with the carrier polymer of the carrier member. Excipients can provide for facilitated or controlled release of the therapeutic agent upon exposure to fluid environments (such as the environment within the small intestine); can provide for stabilization of the therapeutic agent against physical, chemical and/or thermal stressors, for example during processing, manufacture of the system, storage, or use; can enhance transport of the therapeutic agent across the gastrointestinal wall, such as past or through cellular membranes of the endothelial tissue; or can extend the residence time of the therapeutic agent at the intestinal wall.

The therapeutic agent can be a small molecule drug or a biomolecule, such as a polypeptide (which may be a single chain polypeptide or may include two or more separate interacting polypeptide chains) or a polynucleotide (which may be a single-stranded polynucleotide or a double stranded polynucleotide). In some embodiments, the polynucleotide is DNA (which may be single stranded DNA or double stranded DNA), RNA (which may be single-stranded RNA or double-stranded RNA), or a nucleic acid derivative such as a peptide nucleic acid (PNA). Exemplary therapeutic agents include, but are not limited to, natural polypeptides, synthetic (e.g., recombinant or mutant) polypeptides, modified peptides, nucleotides, modified nucleotides, oligonucleotides, RNAi, mRNA, antisense oligonucleotides, CpG DNA, siRNA, miRNA, an aptamer, modified oligonucleotides, plasmids, small molecules, natural products, synthetic analogs of natural products modified natural products, proteins, modified proteins, or a mopholino. The polypeptide can include, for example, 3, 4, 5, 6, 7, 8, 9, about 10 or more, about 15 or more, about 20 or more, about 25 or more, about 30 or more, about 40 or more, about 50 or more, about 75 or more, about 100 or more, or about 150 or more amino acids. For example, in some embodiments, the polypeptide includes about 3 to about 500 amino acids, such as about 3 to about 10, about 10 to about 15, about 15 to about 20, about 20 to about 25, about 25 to about 30, about 30 to about 40, about 40 to about 50, about 50 to about 75, about 75 to about 100, about 100 to about 150, about 150 to about 250, or about 250 to about 500 amino acids. In some embodiments, the polypeptide is a signaling polypeptide, an enzyme, or an antibody or fragment thereof. The polynucleotide can include, for example 3, 4, 5, 6, 7, 8, 9, about 10 or more, about 15 or more, about 20 or more, about 25 or more, about 30 or more, about 40 or more, about 50 or more, about 75 or more, about 100 or more, or about 150 or more nucleotides. For example, in some embodiments, the polynucleotide includes about 3 to about 500 amino acids, such as about 3 to about 10, about 10 to about 15, about 15 to about 20, about 20 to about 25, about 25 to about 30, about 30 to about 40, about 40 to about 50, about 50 to about 75, about 75 to about 100, about 100 to about 150, about 150 to about 250, or about 250 to about 500 nucleotides.

In some embodiments, the carrier member or the coating on the carrier member comprising the therapeutic agent includes an excipient configured to facilitate or control release in the enteric environment. Examples include solubilizes, surfactants, wetting agents, salts, lipids, non-ionic surfactants, cationic surfactants, anionic surfactants zwitterionic surfactants, polysorbates, polyethers, simple sugars, complex sugars, complex carbohydrates, buffers, ion-pairing agents, alkylglycosides, hydrophilic polymers (natural or synthetic), or amphiphilic polymers (natural or synthetic). Other excipients configured to facilitate or control release in the enteric environment can include ionizable agent, such as ionizable lipids or polymers. Ionizable agents include one or more moieties having a pKa in a biorelevant range (i.e., between 4 and 10). In some embodiments, the ionizable agent has one or more moieties having a pKa between 4 and 5, between 5 and 6, between 6 and 7, between 7 and 8, between 8 and 9, or between 9 and 10. Example ionizable lipids include D-Lin-DMA, D-Lin-DAP, D-Lin-K-DMA, D-Lin-KC2-DMA, D-Lin-KC3-DMA, D-Lin-KC4-DMA, D-Lin-MC3-DMA, and other ionizable lipidoids.

In some embodiments, the carrier member or the coating on the carrier member comprising the therapeutic agent includes a protective excipient. Protective excipients can stabilize the therapeutic agent during storage and upon exposure to the gastrointestinal environment. These may include lyoprotectants, humectants, cryoprotectants, water-replacement polyols, ethers, esters, antioxidants, chelating agents, sacrificial reducing agents, buffering agents, crosslinked gels that reduce molecular mobility, and inhibitors of enzymatic degradation (such as protease inhibitors). Antioxidants and sacrificial reducing agents may include hydrophobic agents such as d-alpha tocopherol and its derivatives, hydrophilic agents such as amino including methionine, vitamins such as ascorbic acid, and other common agents. Chelating agents include EDTA, citric acid, and polyionic agents such as polyhistidine. Inhibitors of enzymatic degradation (such as protease inhibitors) may include among others, metal-chelating agents, trypsin inhibitors (for example, soybean trypsin inhibitor), aprotinin, puromycin, serpin, camostat mesilate, chromostatin, ovomucoids, bacitracin, or polymer inhibitor conjugates (such as carbosymethl cellulose-clastinal).

In some embodiments, a permeability enhancing agent (such as a muco-adhesive agent, a muco-permeating agent, a cell membrane permeation enhancer, or a cell junction permeation enhancer) is included in the carrier member with the therapeutic drug or the coating containing the therapeutic drug. Mucoadhesive agents can include agents that preferentially associate at the GI wall through charged/electrostatic affinity, structural affinity, or bulk partitioning. Examples include chitosan, sodium carboxymethyl cellulose, hydroxy ethylcellulose, alginate, poly(methacrylic acid), poloxamer, polyvinylpyrrolidone and polyacrylic acid. Muco-permeating agents may act by promoting compatibility of the dosage form surface or its delivered agent(s) with the mucus or by reducing the integrity of the mucus layer. Example muco-penetrating agents include polyethylene glycol and block co-polymers of polyethylene glycol with other synthetic biocompatible polymers such as poly lactide-co-glycolides or natural polymers such as alginates or chitosan. Cell membrane and tight-junction permeation enhancers may be utilized to facilitate the transport of active agents into or past the cell surface, enhancing uptake and bioavailability of the active agent. Additional examples of permeability enhancing agents include fatty acids (such as C8, C10 and C12 fatty acids, for example caprylate, caprate, and laurate and their salts), bile salts, chitosan, surfactants, glycerides, steroidal detergents, acylcarnitines, alkanoylcholines, N-acetylated-a-amino acids, N-acetylated non-a-amino acids, and thiolated polymers. Cell penetrating peptides may also be used.

In some embodiments, the therapeutic agent is formulated within liposomes, nanoparticles (such as nano-liposomes or solid-lipid nanoparticles), or self-emulsifying systems, which can provide for enhanced transport and delivery. Such multi-molecular constructs can be prepared with the agent and entrapped or embedded within the dosage form or can be prepared by dispersing the agents within the dosage form for in-situ formation of the multi-molecular structure at the time of use due to self-association or induced self-association.

The carrier member with the therapeutic agent or the coating containing the therapeutic agent can include one or more porogens, disintegrants, or osmotic agents, which can promote hydration and/or release of the active agents. Exemplary porogens are described elsewhere herein, and can include, but are not limited to, sugars, salts, enteric polymers, and hydrophilic polymers. Disintegrants may include the same as well as crosslinked, high molecular weight, or insoluble agents such as crosspovidone or sodium starch glycolate. Osmotic agents generally consist of salts and sugars and other low molecular weight agents.

Additional enteric materials (such as enteric polymers) may be included in a coating containing the therapeutic agent, which can promote release of the therapeutic agent in the small intestine and limit release of the therapeutic agent within the gastric environment. Exemplary enteric materials can include, hydroxypropyl methyl cellulose (such as hydroxyl methyl cellulose acetate succinate (HPMCAS) or hydroxypropyl methylcellulose phthalate), shellac, cellulose acetate phthalate, polymethacrylates, cellulose acetate trimellitate, and poly(vinyl acetate pthalatae).

Plasticizers may be incorporated with the therapeutic agent in the carrier member or coating to provide flexibility during processing, storage, and application. As the system includes flexible and/or elastomeric agents, the therapeutic agent may need to be retained in a matrix that is able to withstand conformational change, or to provide or enhance the flexibility of the dry or hydrated matrix. Plasticizers that can be used include the classes of phthalates, phosphates, citrates, tartrates, adipates, sebacates, sulfonamides, succinates, glycolates, glycerolates, or low molecular weight polyethylene glycol, benzoates, myristates, and halogenated phenyls. Specific plasticizers that can be used include triacetin, triethyl citrate (TEC), PEG, poloxamer, tributyl citrate, and dibutyl sebacate.

The coating containing the therapeutic agent may be applied to the entire system (including any carrier members, linkers and/or central members of the system), or to a portion of the system. For example, in some embodiments, the coating is applied only to the carrier members. In some embodiments, the coating is applied only to a portion of the carrier members, such as the distal ends of the carrier members in a stellate design or the outer portion or outer surface of the system in a ring shape design. The outer surface in a ring shape design refers to the portion or surface distal from the central opening.

The coating containing the therapeutic agent can be about 10 μm to about 300 μm thick (such as about 10 μm to about 20 μm thick, about 20 μm to about 30 μm thick, about 30 μm to about 40 μm thick, about 40 μm to about 50 μm thick, about 50 μm to about 75 μm thick, about 75 μm to about 100 μm thick, about 100 μm to about 150 μm thick, about 150 μm to about 200 μm thick, or about 200 μm to about 300 μm thick). The coating by include a swellable material, such as a hydrogel, which when hydrated in the small environment can swell to increase the thickness of the coating.

By way of example, the system may be coated by dipping, rolling, spraying, or otherwise contacting a liquid or gel containing the therapeutic agent (and one or more excipients, if present) in one or more steps to apply the therapeutic agent to the surface of the system. The resulting coating may be solidified onto the system (or carrier members form through dehydration, pH-induced condensation, crosslinking, or other curing process. During application, the therapeutic agent may be dissolved, emulsified, or suspended in a solvent, which may be aqueous or organic based. The therapeutic agent may be dissolved or suspended in the presence of excipients that enhance the solubility or suspension stability and one or more of said excipients become incorporated as part of the coating. Upon drying, the coat is substantially of stable physical form and is located in whole or in most part on the surfaces of the dosage form that may be in direct contact with the intestinal membrane.

The system, whether the therapeutic agent is within a carrier member or coated on the carrier member, can further include one or more additional coating layers. The additional coating layers are generally on top of any coating with the therapeutic agent. The one or more additional coatings can include, for example, a release-modifying coating, a protective coating (which may be, for example, an enteric coating or an anti-enteric coating), a muco-adhesive coating, or an anti-self-adhesive coating. For example, a system may be coated with an enteric coating (such as HPMC or HPMCAS) to protect the therapeutic agent from the gastric environment to prevent or reduce release of the drug before the system enters the small intestine. Once in the small intestine, the enteric coating dissolves or degrades, and the therapeutic agent can be released in the small intestine. Using the enteric coating the system can expand from the compacted configuration to the extended configuration within the stomach and pass through the pylorus without substantial release of the therapeutic agent until reaching the small intestine. In some embodiments, less than about 5%, less than about 4%, less than about 3%, less than about 2%, or less than about 1% of the therapeutic agent is released from the system before the system reaches the small intestine.

In some embodiments, the system can include a reverse-enteric coating (which may be on top of the enteric coating). A reverse enteric coating can dissolve or degrade within the gastric environment, and inclusion on the reverse-enteric coating can prevent or limit esophageal release of the therapeutic agent.

An exemplary anti-self-adhesive coating can include, for example, talc, which acts to prevent the system from adhering to itself when in the compacted configuration.

### Packaged Enteric Delivery Systems

The enteric delivery systems described herein can be packaged in an orally administrable container, such as a capsule. In some embodiments, the capsule is an enteric capsule, and is configured to release the system in the small intestine. In some embodiments, the capsule is a reverse enteric capsule, and is configured to release the system in the stomach.

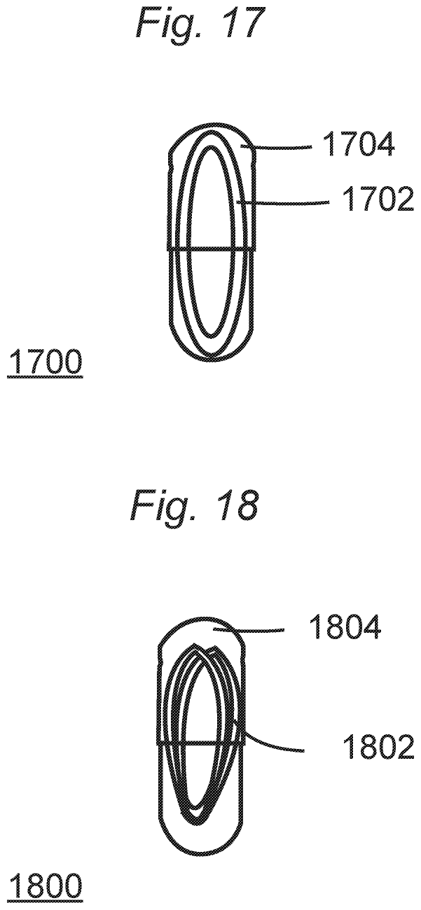

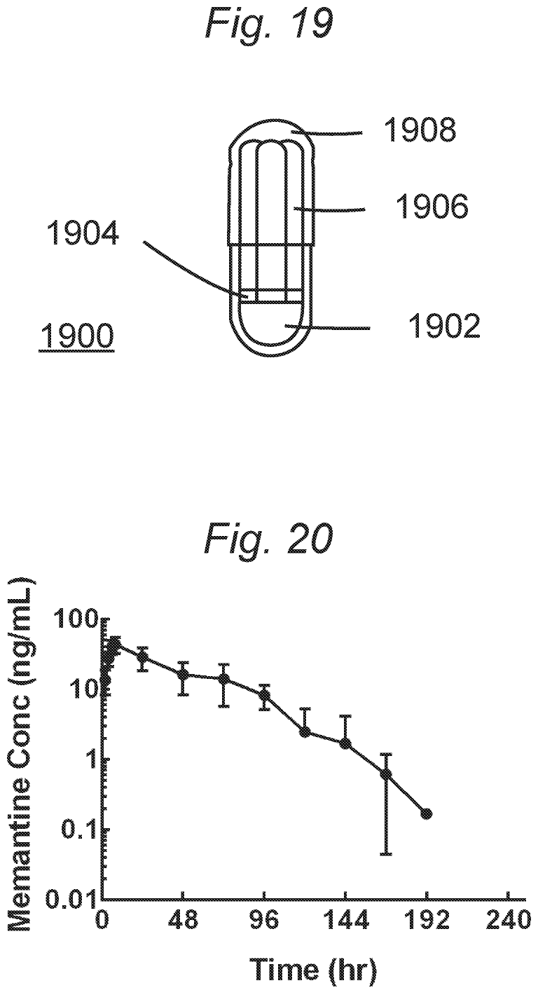

When the system is within the container (e.g., capsule), the system is configured in a compacted configuration. Release from the vehicle allows the system to expand into the expanded configuration. FIG. 17 illustrates a toroidal enteric delivery system in a capsule. FIG. 18 illustrates a toroidal enteric delivery system which is folded in two to further compact the system, in a capsule. FIG. 19 illustrates a stellate enteric delivery system in a compacted configuration in a capsule.

### Methods of Administering a Therapeutic Agent

A therapeutic agent can be administered to a patient (such as a human patient) by orally administering to the patient an enteric delivery system in a compacted configuration; expanding the enteric delivery system to an expanded configuration; applying, using the expanded enteric delivery system, outwardly directed pressure to the intestinal wall of the small intestine of the patient; and releasing the therapeutic agent from enteric delivery system to transport the therapeutic agent across the enteric mucosa of the small intestine. The enteric delivery includes comprising one or more carrier members (which may be an elongated carrier member) comprising a carrier polymer and the therapeutic agent. The enteric delivery system can be, for example, any of the enteric delivery systems described herein.

In some embodiments, the enteric delivery system is expanded within the small intestine of the patient, such as the duodenum.

In some embodiments, the enteric delivery system is expanded within the stomach of the patient and passes through the pylorus of the patient into the small intestine without substantial release of the therapeutic agent until the system enters the small intestine.

In some embodiments, at least a portion of the system loses structural integrity after a period of time within the small intestine to release the outwardly directed pressure.

In some embodiments, the outwardly directed pressure is released after about 1 to about 72 hours after the system enters the small intestine.

In some embodiments, release of the outwardly directed pressure allows the enteric delivery system to pass through the small intestine.

In some embodiments, the therapeutic agent is a polypeptide or a polynucleotide. In some embodiments, the therapeutic agent is a polypeptide comprising 10 or more amino acids. In some embodiments, the therapeutic agent is a polynucleotide comprising 10 or more nucleotides.

### EXAMPLES

### Example 1: Enteric Delivery System Placement

Multiple animals will be administered with either the ring-shaped or the stellate-shaped enteric delivery system to investigate the probability and location of the enteric delivery device at specified time points.

The systems can be formulated to facilitate in vivo imagining. For example, in a stellate system, stainless steel fiducials (e.g. beads) can be placed along the polymeric drug arms or at the tips of the arms during polymerization of the arms. Alternatively, radioactive tracers (such as barium tracers) can be embedded in the stellate arms of the system or included in the coating. In some examples, the systems can be administered to Yorkshire swine (35-50 kg) or dogs under sedation and through an endoscopic overtube into the duodenum. Serial radiographs will be obtained from multiple positions (including anteroposterior, left lateral and right lateral positions) of the chest, abdomen, and pelvis.

Serial radiographs will be taken after duodenal delivery for up to 15 minutes to confirm deployment from the outer capsule and/or restraining system. Radiographs will then be obtained daily for the next 4 days and three times weekly after the first 5 days. Location and longevity of the enteric delivery system in the duodenum can be confirmed from multiple radiographic views.

### Example 2: Duodenal Placement of a Stellate Enteric Delivery System in Dogs

Six male beagle dogs (each weighing ˜10 kg) were anesthetized and intubated, and stellate-shaped systems bearing memantine drug arms were placed endoscopically through the pylorus into the duodenum. Each stellate system contained steel beads at the tips of the drug arms which facilitated X-ray imaging for confirming successful duodenal placement.

X-rays were collected daily to monitor residence time in the small intestine. Blood samples were collected through Day 10 of the study and processed to plasma for quantitation of memantine using an LC-MS/MS assay. The dogs were monitored for the duration of the study for safety.

The average time to stellate excretion after duodenal placement was 5.0±2.9 days with a range of 1-9 days. There were no clinical observations noted as a consequence of duodenal placement of stellates for the duration of the study that would indicate a safety concern from residence of stellates in the small intestine.

Memantine bioanalysis in plasma collected from dogs during the study showed good exposure from the small intestine with a Tₘₐₓ at 8 hours and sustained release of memantine was measurable for 7 days, as shown in FIG. 20.

### Example 3: Administration of a Toroidal System for Enteric Delivery to an Animal

### Administration and Duodenal Deployment

To assess particular formulations that were developed for ability to achieve enteric drug delivery, a toroidal enteric delivery system as described herein will be administered to a large animal model, such as a dog or a pig. The therapeutic agent will be a protein or a nucleic acid, which is coated on an outer portion of the toroidal system. The coating further includes a permeability enhancing agent, such as sodium caprate. Animals can be anesthetized using conventional means, such as with Telazol and Xylazine. (or alternatively with ketamine or isoflurane) and an endoscopic overtube will be placed under endoscopic visual guidance during intubation into the esophagus, the stomach, and/or through to pylorus into the duodenum. Gelatin capsules containing the structures can be administered via the overtube into the esophagus, stomach and/or into the duodenum directly. The overtube will subsequently be retracted. Serial x-rays will be obtained immediately after delivery to the duodenum to document the process of deployment from the gelatin capsule.

### Determining Sustained Release Profile/Pharmacokinetics

To determine the drug release profiles in animals, blood samples will be withdrawn periodically from the animals receiving the enteric residence device. For example, blood samples will be drawn from the individual at approximately 0 min, 15 min, 30 min, 1 hr, 2 hr, 4 hr, 8 hr, 16 hr, 24 hr, after the enteric residence system has deployed. Further blood samples will be drawn from the animal daily up to 14 days after the enter residence system has initially deployed.

The drug levels will be quantified using liquid chromatography (LC) or with liquid chromatography-mass spectrometry (LC-MS) and plotted for drug release profiles over time. The time when the maximum plasma concentration is reached after placement of the system will be noted as Tₘₐₓ.

### Example 4: Administration of a Stellate System for Enteric Delivery to an Animal

### Administration and Duodenal Deployment

To assess particular formulations that were developed for ability to achieve enteric drug delivery, a stellate-shaped enteric delivery system as described herein will be administered to a large animal model, such as a dog or a pig. The therapeutic agent will be a protein or a nucleic acid, which is coated on the distal portions of the arms of the stellate-shaped system. The coating further includes a permeability enhancing agent, such as sodium caprate. Animals can be anesthetized using conventional means, such as with Telazol and Xylazine, (or alternatively with ketamine or isoflurane) and an endoscopic overtube will be placed under endoscopic visual guidance during intubation into the esophagus, the stomach, and/or through to pylorus into the duodenum. Gelatin capsules containing the structures can be administered via the overtube into the esophagus, stomach and/or into the duodenum directly. The overtube will subsequently be retracted. Serial x-rays will be obtained immediately after delivery to the duodenum to document the process of deployment from the gelatin capsule.

### Determining Sustained Release Profile Pharmacokinetics

To determine the drug release profiles in animals, blood samples will be withdrawn periodically from the animals receiving the enteric residence device. For example, blood samples will be drawn from the individual at approximately 0 min, 15 min, 30 min, 1 hr, 2 hr, 4 hr, 8 hr, 16 hr, 24 hr, after the enteric residence system has deployed. Further blood samples will be drawn from the animal daily up to 14 days after the enter residence system has initially deployed.

The drug levels will be quantified using liquid chromatography (LC) or with liquid chromatography-mass spectrometry (LC-MS) and plotted for drug release profiles over time.
The time when the maximum plasma concentration is reached after placement of the system will be noted as Tₘₐₓ.

What is claimed is:

1. A system for enteric delivery of a therapeutic drug, comprising:
one or more carrier members comprising a carrier polymer and a therapeutic agent,
the system configurable in a compacted configuration and an expanded configuration, wherein the system is configured to (1) expand from the compacted configuration to the expanded configuration within the small intestine, or (2) expand from the compacted configuration to the expanded configuration within the stomach and pass through the pylorus without substantial release of the therapeutic agent until reaching the small intestine;
wherein the system is sized to maintain contact with the intestinal wall of the small intestine by applying an outwardly directed pressure to the intestinal wall and transport at least a portion of the therapeutic agent across the enteric mucosa of the small intestine; and
wherein at least a portion of the system loses structural integrity after a period of time within the small intestine to release the outwardly directed pressure.

2. The system of claim 1, wherein the system is configured to expand from the compacted configuration to the expanded configuration within the small intestine.

3. The system of claim 1, wherein the system is configured to expand from the compacted configuration to the expanded configuration within the stomach and pass through the pylorus without substantial release of the therapeutic agent until reaching the small intestine.

4. The system of any one of claims 1-3, wherein the one or more carrier members comprise a coating comprising the therapeutic agent.

5. The system of claim 4, wherein the coating further comprises a permeability enhancing agent.

6. The system of any one of claims 1-3, wherein the therapeutic agent is loaded into the carrier polymer.

7. The system of claim 6, wherein a permeability enhancing agent is loaded into the carrier polymer.

8. The system of claim 5 or 7, wherein the permeability enhancing agent is a muco-adhesive agent or a muco-permeating agent.

9. The system of claim 8, wherein the permeability enhancing agent is a fatty acid, a bile salt, chitosan, a thiolated polymer, or a cell penetrating peptide.

10. The system of any one of claims 1-9, wherein the outwardly directed pressure is released after about 1 hour to about 72 hours after the system enters the small intestine.

11. The system of any one of claims 1-10, wherein release of the outwardly directed pressure allows for passage of the carrier members through the small intestine.

12. The system of any one of claim 1-11, wherein the system is configured to transport the therapeutic agent across the enteric mucosa for about 1 hour to about 72 hours.

13. The system of any one of claims 1-12, wherein the system is sized to maintain contact with the intestinal wall of the duodenum by applying an outwardly directed pressure to the intestinal wall of the duodenum and transport at least a portion of the therapeutic agent across the enteric mucosa of the duodenum.

14. The system of any one of claims 1-13, wherein the one or more carrier members comprise a hollow core.

15. The system of any one of claims 1-14, wherein the one or more carrier members comprise a solid core.

16. The system of any one of claims 1-15, wherein the one or more carrier members are configured to lose structural integrity after a period of time within the small intestine to release the outwardly directed pressure.

17. The system of claim 16, wherein the one or more carrier members are configured to lose structural integrity through erosion, degradation, or softening of the one or more carrier members.

18. The system of any one of claims 1-16, wherein the one or more carrier members are arranged in a ring shape.

19. The system of any one of claims 1-18, further comprising one or more linkers that join the one or more carrier members to form a ring shape, the one or more linkers comprising a polymer configured to lose structural integrity after a period of time in the small intestine.

20. The system of claim 19, wherein the therapeutic drug is within a coating on or in an outer portion of the ring shape, but not on or in an inner portion of the ring shape.

21. The system of any one of any one of claims 1-17, wherein the system further comprises an elastomeric central member attached to a plurality of arms radiating outwardly from the central member when the system is in an extended configuration, the arms comprising one or more carrier members.

22. The system of claim 21, wherein the therapeutic drug of the system is preferentially disposed on or within distal ends of the arms relative to the elastomeric central member.

23. The system of claim 22, wherein the elastomeric central member comprises a polymer configured to lose structural integrity after a period of time in the small intestine.

24. The system of claim 23, wherein the elastomeric central member is configured to lose structural integrity through erosion, degradation, or softening of the elastomeric central member.

25. The system of any one of claims 21-24, wherein the elastomeric central member is joined to the arms through one or more linkers comprising a polymer configured to lose structural integrity after a period of time in the small intestine.

26. The system of claim 19, 20, or 25, wherein the system loses structural integrity through erosion, degradation, or softening of the one or more linkers.

27. The system of any one of claims 1-26, wherein the carrier members have a circular, elliptical, or teardrop cross section.

28. The system of any one of claims 1-27, wherein the therapeutic agent is a polypeptide or a polynucleotide.

29. The system of claim 28, wherein the therapeutic agent is a polypeptide comprising 10 or more amino acids.

30. The system of claim 28, wherein the therapeutic agent is a polynucleotide comprising 10 or more nucleotides.

31. The system of any one of claims 1-30, wherein the small intestine is a small intestine of a human.

32. The system of any one of claims 1-31, wherein the system is coated with a protective coating.

33. The system of claim 32, wherein the protective coating is an enteric coating.

34. The system of claim 32 or 33, wherein the system is further coated with a reverse-enteric coating.

35. A therapeutic dosage form comprising a capsule encapsulating the system of any one of claims 1-34.

36. The therapeutic dosage form of claim 35, wherein the capsule is an enteric capsule.

37. A method of administering a therapeutic agent to a patient, comprising:
orally administering to the patient an enteric delivery system in a compacted configuration, the enteric delivery system comprising one or more carrier members comprising a carrier polymer and the therapeutic agent;
expanding the enteric delivery system to an expanded configuration;
applying, using the expanded enteric delivery system, outwardly directed pressure to the intestinal wall of the small intestine of the patient; and
releasing the therapeutic agent from enteric delivery system to transport the therapeutic agent across the enteric mucosa of the small intestine.

38. The method of claim 37, wherein the enteric delivery system is expanded within the small intestine.

39. The method of claim 37 or 38, wherein the enteric delivery system expands in the duodenum of the patient.

40. The method of claim 37, wherein the enteric delivery system is expanded within the stomach of the patient and passes through the pylorus of the patient into the small intestine without substantial release of the therapeutic agent until the system enters the small intestine.

41. The method of any one of claims 37-40, wherein at least a portion of the system loses structural integrity after a period of time within the small intestine to release the outwardly directed pressure.

42. The method of claim 41, wherein the outwardly directed pressure is released after about 1 to about 72 hours after the system enters the small intestine.

43. The method of claim 41 or 42, wherein release of the outwardly directed pressure allows the enteric delivery system to pass through the small intestine.

44. The method of any one of claims 37-43 wherein the therapeutic agent is a polypeptide or a polynucleotide.

45. The method of claim 44, wherein the therapeutic agent is a polypeptide comprising 10 or more amino acids.

46. The method of claim 44, wherein the therapeutic agent is a polynucleotide comprising 10 or more nucleotides.

47. The method of any one of claims 37-46, wherein the enteric delivery system is the system according to any one of claims 1-34.

48. The method of any one of claims 37-46, wherein the patient is a human.
